(19) United States
(12) Patent Application Publication
(10) Pub. No.: US 2021/0262012 A1
(43) Pub. Date: Aug. 26, 2021

(54) METHODS FOR THE COMPREHENSIVE IDENTIFICATION OF ANTIMICROBIAL RESISTANCE MARKERS BY SEQUENCING

(71) Applicant: Ares Genetics GmbH, Vienna (AT)

(72) Inventors: Andreas Posch, Vienna (AT); Jonas Ramoni, Vienna (AT); Stephan Beisken, Vienna (AT); Achim Plum, Berlin (DE)

(21) Appl. No.: 16/762,303

(22) PCT Filed: Nov. 7, 2018
(86) PCT No.: PCT/EP2018/080517

§ 371 Date: May 7, 2020

(30) Foreign Application Priority Data
Nov. 10, 2017 (EP) 17 201 102.5

(51) Int. Cl. C12Q 1/689

(52) U.S. Cl. CPC C12Q 1/689; C12Q 2600/106; C12Q 2600/156

(57) ABSTRACT

The present invention relates to the systematic identification of antimicrobial resistance markers (biomarkers) in a microorganism for a particular compound and the use of such identified markers for the screening of microorganisms for antimicrobial resistance, as well as for screening/predicting antimicrobial compounds that can overcome the resistance provided by one or more of the antimicrobial resistance marker(s).

### 1. FIELD OF THE INVENTION

The present invention relates to the systematic identification of antimicrobial resistance markers (biomarkers) in a microorganism for a particular compound and the use of such identified markers for the screening of microorganisms for antimicrobial resistance, as well as for screening/predicting antimicrobial compounds that can overcome the resistance provided by the one or more of the antimicrobial resistance marker(s).

### 2. BACKGROUND OF THE INVENTION

Antimicrobial resistance is spreading around the world and it is estimated that infections caused by drug-resistant microorganisms cause over 700,000 deaths every year (O'Neill, 2014, Antimicrobial Resistance: Tackling a Crisis for the Health and Wealth of Nations, https://amr-review.org/sites/default/files/AMR %20Review %20Paper%20-%20Tackling %20a %20crisis %20 for %20the %20health %20 and %20wealth %20of%20nations_1.pdf). A variety of molecular mechanisms contribute to the development of drug-resistance, including genomic mutations such as single nucleotide polymorphisms and horizontal resistance-gene transfer from surrounding microorganisms (Blair et al., 2015, Nat Rev Micro 13:42-51; Von Wintersdorff et al., 2016, Frontiers in Microbiology 7:173).

Detailed knowledge about these genetic mutations leading to resistance is of great interest to the diagnostic industry since they serve as diagnostic markers (biomarkers). Additionally, a detailed understanding of the causes and mechanisms of antimicrobial resistance helps the pharmaceutical industry with the prediction of the likelihood that microorganisms develop resistance mechanisms against compounds or compound combinations which are either in clinical development or approved and on the market. Furthermore, the comprehensive integration of markers and combinations thereof in molecular diagnostics systems allows for better informed treatment decisions, thus preserving compound efficacy, e.g., minimizing the development of resistant strains, and improving product lifecycle management.

The evolution of drug-resistant microorganisms can be studied by their cultivation on media containing lethal or non-lethal concentrations of the compound of interest (Gullberg et al., 2011, PLoS Pathogens 7:1-9, Hughes and Andersson, 2012, Current Opinion in Microbioloy 15:555-560). However, this alone is not sufficient to comprehensively understand antimicrobial resistance (AMR) for a particular compound because it ignores the acquisition of resistance genes via horizontal evolution. For example, microorganisms exposed to compounds like teixobactin show no demonstrable intrinsic or spontaneous vertical evolution of resistance mechanisms (Ling et al., 2015, Nature 517:455-459). Thus, there remains a need in the art for methods that allow for the comprehensive detection of resistance biomarkers for a particular compound in a microorganism. The present invention fulfills such a need.

### 3. SUMMARY OF THE INVENTION

The present invention relates to a method for the comprehensive detection of nucleic acid encoded antimicrobial resistance biomarkers associated with a particular compound in a microorganism, including the detection and evaluation of evolutionary resistance mechanisms caused by a single mutation or a series of single-step mutations, as well as the detection of those resistance mechanisms caused by horizontal gene transfer from the surrounding microbial community.

Accordingly, the present invention is directed to a method for providing a panel of nucleic acid-encoded antimicrobial resistance (AMR) biomarkers of a microorganism, wherein the panel of AMR biomarkers relates to a compound having antimicrobial activity, the method comprising:

I (a) culturing the microorganism in the presence of the compound, wherein the compound is present in an amount that does not substantially inhibit wild type growth of the microorganism under the same culture conditions absent the compound, and
(b) determining the genetic profile of the cultured microorganism obtained in step (a);

II (c)(i) culturing the microorganism in the presence of the compound, wherein the compound is present in an amount that substantially inhibits wild type growth of the microorganism under the same culture conditions absent the compound, and (ii) selecting a cultured microorganism whose growth in the presence of the compound is substantially the same as in the absence of the compound, and
(d) determining the genetic profile of the selected microorganism obtained in step (c); and

III (e)(i) culturing the microorganism in the presence of the compound, wherein the compound is present in an amount that substantially inhibits wild type growth of the microorganism under the same culture conditions absent the compound, and (ii) selecting a cultured microorganism whose growth in the presence of the compound is substantially the same as in the absence of the compound, wherein the microorganism is a recombinant microorganism, and
(f) determining the genetic profile of the selected microorganism obtained in step (e);

wherein the genetic profiles determined in steps (b), (d), and (f) are each compared to a reference genetic profile of the microorganism to identify a panel of nucleic acid-encoded antimicrobial resistance (AMR) biomarkers relating to the compound.

In an embodiment, the genetic profile is determined by sequencing the genomic and optionally the extra-genomic nucleic acids of the microorganisms obtained in steps (a), (c), and (e), e.g., by next-generation sequencing. In an embodiment, the extra-genomic nucleic acids are naturally occurring in the microorganism or are not naturally occurring, for example, an artificial plasmid transformed into the microorganism. In preferred embodiments of the invention, the sequencing is performed by molecular high-throughput sequence analysis, i.e., by next-generation or third generation sequencing, such as by the Illumina/Solexa or the Oxford Nanopore methodology.

In an embodiment, the biomarker identified according to the present invention can be a mutation in the sequence of an encoding nucleic acid of the microorganism, e.g., a point mutation, i.e., a base substitution, or an insertion or deletion of one or more bases, or is the result of a fusion of two encoding nucleic acid sequences, which mutation results in the alteration of the encoded nucleic acid or amino acid sequence.

In an embodiment, after step (a) and prior to step (b), the method can further comprise (i) culturing the microorganism in the presence of the compound in an amount that substantially inhibits wild type growth of the microorganism under the same culture conditions absent the compound, and (ii) selecting a cultured microorganism whose growth in the presence of the compound is substantially the same as in the absence of the compound. In an embodiment, the steps (i) and (ii) are repeated at least once. In other embodiments, the steps (i) and (ii) are repeated twice, three times (3×), 4×, 5×, 6×, 7×, 8×, 9×, or at least 5 times or at least 10 times.

In an embodiment, the culturing step (a) can take place over an extended period of time, i.e., for more than 1 or 2 days, for example, more than 2, 3, 4, 5, 6, 7, 8, 9 or 10 days, or for 1 to several weeks, e.g., 1, 2, 3, 4, 5, 6, 7, 8, 9, 10 or more weeks, or for a period of months, e.g., 1, 2, 3, 4, 5, 6, 7, 8, 9, or 10 months.

In an embodiment, the microorganism can be a bacterium, a fungus, e.g., a filamentous fungus, or a parasite, preferably a bacterium. In an embodiment, the microorganism can be a recombinant microorganism which is genetically modified, for example, to comprise an expression library. The expression library can be derived from a different strain of the same microorganism or is derived from a different microorganism or is derived from a consortium of microorganisms, i.e., derived from “donor” microorganism(s).

In an embodiment, culturing takes place on a solid surface or in liquid culture at conditions conducive for the growth of the microorganism. In an embodiment, the growth of the microorganism is measured by cell doubling time, which can be determined by methods known in the art such as counting the number of microorganism directly or indirectly (light absorbance) or by determining over time the presence and/or amount of nucleic acid, e.g., DNA, or a marker peptide/polypeptide, or a nutrient or other compound produced or consumed by the microorganism.

In an embodiment, the compound is a compound known to inhibit growth of the microorganism, such as a known antibiotic or antifungal compound.

In an embodiment, the method further comprises testing at least one biomarker of the identified panel of biomarkers for the ability to confer antimicrobial resistance to the compound. Preferably, the testing comprises culturing a microorganism recombinantly expressing the at least one biomarker in the presence of the compound in an amount that substantially inhibits wild-type growth of the microorganism not having the biomarker. If the microorganism recombinantly expressing the biomarker is able to grow in the presence of the compound, then the biomarker at least contributes to the ability of the microorganism to be resistant to the compound.

Further, the present invention is directed to a panel of nucleic acid-encoded antimicrobial resistance biomarkers identified by the methods for providing a panel of nucleic acid-encoded antimicrobial resistance (AMR) biomarkers of a microorganism described herein.

The present invention is directed to a method for determining/screening for the presence of a microorganism resistant to a particular compound in a host organism, comprising detecting at least one nucleic acid-encoded antimicrobial resistance biomarker identified in a microorganism obtained from the host organism, preferably wherein the biomarker is identified by the methods for providing a panel of nucleic acid-encoded antimicrobial resistance (AMR) biomarkers of a microorganism described herein. In an embodiment, the host organism is a mammal, preferably a human.

### 4. DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Kölbl, Eds., (1995) Helvetica Chimica Acta, CH-4010 Basel, Switzerland.

The practice of the present invention will employ, unless otherwise indicated, conventional methods of biochemistry, cell biology, immunology, and recombinant DNA techniques which are explained in the literature in the field (cf., e.g., Molecular Cloning: A Laboratory Manual, 4th Edition, M. R. Green, J. Sambrook et al. eds., Cold Spring Harbor Laboratory Press, Cold Spring Harbor 2012).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e., the subject-matter consists in the inclusion of a stated member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

The present invention, inter alia, allows for the comprehensive determination of nucleic acid-encoded antimicrobial resistance biomarkers (plurality of biomarkers) which are relevant for the resistance of a particular microorganism to a particular compound, e.g., antibiotic. In context of the present invention, the term “antimicrobial resistance” or “antibiotic resistance” means a loss of susceptibility of microorganisms to the killing, or growth-inhibiting properties of an antibiotic agent. It also relates to resistance of a microorganism to an antimicrobial drug that was originally effective for treatment of infections caused by it. Resistant microorganisms, including bacteria, fungi, viruses and parasites, are able to withstand attack by antimicrobial drugs, such as antibacterial drugs, antifungals, antivirals, and anti-malarials, so that standard treatments become ineffective and infections persist.

The determination of the plurality of biomarkers encompasses growing/culturing a wild-type microorganism, i.e., one that is not resistant to a compound, in the presence of the compound separately at concentrations of the compound that substantially and do not substantially inhibit the wild-type growth of the microorganism, as well as growing/culturing the wild-type microorganism that has been made recombinant, preferably by the transformation of an expression library into the microorganism, in the presence of the compound at concentrations that substantially inhibit wild-type growth of the recombinant microorganism. After the culturing steps, described in more detail below, the selected microorganisms are collected and processed such that the nucleic acids of the microorganism are sequenced and the sequence information is compared to a reference, which allows for any mutations (preferably in encoding nucleic acids), i.e., biomarkers, to be identified, which mutations confer the ability to grow in the presence of the compound at concentrations that substantially inhibit growth of the microorganism.

By culturing the microorganism under the three separate culture conditions in the presence of the same compound, a comprehensive plurality of nucleic acid-encoded antimicrobial resistance biomarkers can be identified for a particular compound for the particular microorganism. The identified biomarkers can then be used to determine whether or not an isolated microorganism, e.g., from a patient, is resistant to a particular antimicrobial compound.

In certain embodiments where the patient is suffering from an infectious disease cause by the microorganism, the knowledge of resistance can then allow for appropriate treatment with an effective antimicrobial/antibiotic. Moreover, microorganisms isolated from locations where resistance often appears, such as in hospitals and in nursing homes, can be screened for resistance to a particular compound by determining the presence of one or more of the identified plurality of nucleic acid-encoded antimicrobial resistance biomarkers of the microorganism.

In particular, the method of the present invention for providing a panel of nucleic acid-encoded antimicrobial resistance (AMR) biomarkers of a microorganism, wherein the panel of AMR biomarkers relates to a compound having antimicrobial activity, comprises:

I (a) culturing the microorganism in the presence of the compound, wherein the compound is present in an amount that does not substantially inhibit wild type growth of the microorganism under the same culture conditions absent the compound, and
(b) determining the genetic profile of the cultured microorganism obtained in step (a);

II (c)(i) culturing the microorganism in the presence of the compound, wherein the compound is present in an amount that substantially inhibits wild type growth of the microorganism under the same culture conditions absent the compound, and (ii) selecting a cultured microorganism whose growth in the presence of the compound is substantially the same as in the absence of the compound, and
(d) determining the genetic profile of the selected microorganism obtained in step (c); and

III (e)(i) culturing the microorganism in the presence of the compound, wherein the compound is present in an amount that substantially inhibits wild type growth of the microorganism under the same culture conditions absent the compound, and (ii) selecting a cultured microorganism whose growth in the presence of the compound is substantially the same as in the absence of the compound, wherein the microorganism is a recombinant microorganism, and
(f) determining the genetic profile of the selected microorganism obtained in step (e); wherein the genetic profiles determined in steps (b), (d), and (f) are each compared to a reference genetic profile of the microorganism to identify a panel of nucleic acid-encoded antimicrobial resistance (AMR) biomarkers relating to the compound.

Subpart I of the method can be carried out by culturing the microorganism on a growth medium, in the presence of the compound that inhibits growth of the microorganism but at a concentration low enough to allow for substantially wild type (normal) growth of the microorganism. As used herein, not substantially inhibiting the wild-type growth in the presence of a compound means that the growth rate of the wild-type microorganism in the presence of the compound is at least 70%, 75%, 80%, 85%, 90%, 91%, 92%, 93%, 94%, 95%, 96%, 97%, 98%, 99% or more compared to (wild-type) growth under the same conditions absent the compound or that there is no detectable difference in the growth rate between growth in the presence or growth in the absence of the compound. Preferably, the compound is present at a concentration that is a sub-minimal inhibitory concentration. Culturing can take place over hours, days, weeks or months with appropriate passaging/re-plating of the microorganism. Optionally, the cultured microorganism can be transferred to culture conditions where the growth medium contains a concentration of the compound that substantially inhibits the growth of the microorganism to test whether resistance has developed against the compound. As used herein, substantially inhibiting the wild-type growth in the presence of a compound means that the growth rate of the wild-type microorganism in the presence of the compound is no more than and preferably less than 30%, 25%, 20%, 15%, 10%, 9%, 8%, 7%, 6%, 5%, 4%, 3%, 2%, or 1% or less compared to (wild-type) growth under the same conditions absent the compound or is the complete inhibition of growth. Optionally, the microorganism prior to and/or during culturing can be treated to conditions leading to accelerated evolution, i.e., treated with a mutagen such as UV- or X-irradiation or with a chemical mutagen, such as colchicine, ethidium bromide, proflavine, alkylators, including ethyl methane sulfonate (EMS), methyl methane sulfonate (MMS), diethylsulfate (DES), and nitrosoguanidine.

Subpart II of the method can be carried out by culturing the wild-type microorganism on a growth medium, in the presence of the compound at a concentration that substantially inhibits wild type growth of the microorganism. As used herein, substantially inhibiting the wild-type growth in the presence of a compound means that the growth rate of the wild-type microorganism in the presence of the compound is no more than and preferably less than 30%, 25%, 20%, 15%, 10%, 9%, 8%, 7%, 6%, 5%, 4%, 3%, 2%, or 1% or less compared to (wild-type) growth under the same conditions absent the compound or is the complete inhibition of growth. Preferably, the wild-type microorganism does not grow at all in the presence of the compound at the concentration used. Cultured microorganisms (survivors) will then be selected on the basis of being able to grow in the presence of the compound. The rate of growth of the cultured microorganism (survivors) may vary in the presence of the compound compared to its absence since the survivors may have other mutations affecting growth. In an embodiment, the cultured microorganism grows slower in the absence of the compound as compared to the presence of the compound. In an embodiment, the cultured microorganism grows faster in the absence of the compound as compared to the presence of the compound. Preferably, the rate of growth is substantially the same as or is equal to the growth of the wild-type microorganism in the absence of the compound. In an embodiment, the selected microorganism is one that is able to grow in the presence of the compound at any rate of growth in the presence of the compound at the same concentration that prevents the wild-type microorganism from growing. Optionally, the microorganism prior to and/or during culturing can be treated to conditions leading to accelerated evolution, i.e., treated with a mutagen such as UV- or X-irradiation or with a chemical mutagen, such as colchicine, ethidium bromide, proflavine, alkylators, including ethyl methane sulfonate (EMS), methyl methane sulfonate (MMS), diethylsulfate (DES), and nitrosoguanidine.

Those selected microorganisms (survivors) of subpart II, as well as those cultured microorganisms of subpart I, that are able to grow, e.g., at any rate, in the presence of the compound at a concentration that substantially or completely prevents growth of the corresponding wild type microorganism can have a mutation in an encoding nucleic acid (an antimicrobial resistance biomarker), e.g., a spontaneous mutation, that results in a change in the molecule/product encoded by the nucleic acid. This change allows for growth at any rate of growth, preferably at substantially the same or the same rate of growth in the presence of the compound compared to the absence of the compound.

Subpart III of the method is carried out as described for subpart II except that the microorganism is a microorganism that has been recombinantly engineered to comprise donor nucleic acids derived from other microorganisms, e.g., to comprise an expression library, and that the selected microorganisms that are able to grow in the presence of the compound can be due to the presence of an encoded molecule/product expressed from a donor nucleic acid, e.g., from the expression library. Preferably, the culturing conditions/time in subpart III ensures that the ability to grow in the presence of the compound is much more likely due to the presence of the donor nucleic acids, not due to a mutation in the endogenous nucleic acids native to the microorganism. The preferable culturing time will be no more than the time required for the encoded molecule(s) to be expressed and determine the ability of the microorganism to grow in the presence of the compound after the microorganism is exposed to the compound under the appropriate culture conditions. In an embodiment where the recombinant microorganism is already growing, e.g., in log phase, the culturing time is no more than the time required after contacting the compound to determine whether the microorganism stops or does not stop growing in the presence of the compound.

In an embodiment, the donor nucleic acids, such as an expression library, can be obtained commercially or can be generated de novo and can be derived from the nucleic acids obtained from related and/or unrelated microorganisms, e.g., different strains of the same species or from the same genus or from the same family (donor microorganisms). Preferably, the library is derived from a consortium or collection of microorganisms, for example, several strains of several different species of the same genus. Exemplary sources of donor microorganisms, i.e., sources of donor nucleic acids include, but are not limited to, soil, aerosols, lakes and rivers and sediments thereof, water outflows from irrigated fields or from sewage treatment plants, animal faeces or urine obtained in the wild or from domesticated animals, faeces and urine obtained from humans, preferably from hospitalized human patients.

Production of the expression libraries will be done according to methods known in the art. Briefly, nucleic acids from a donor microorganism will be collected and placed into an acceptor site (cloning site) of the backbone of an expression vector, which nucleic acids can be operably linked to promoter sequences which are active in the microorganism into which the expression vector is to be transformed, such that the encoded molecule(s)/product(s) of the inserted nucleic acids is expressed. The backbone of the expression vector also can comprise a resistance gene or an auxotrophic marker, e.g., to ensure that wild type microorganisms transformed with the expression vector will grow only under certain conditions, e.g., to insure that the transformed microorganism will not be pathogenic to humans. To that effect the transformed microorganism also may lack an essential gene, e.g., argA, asnA, asnB, aspC, and tyrB of E. coli and equivalents in other microorganisms. Also, a promoter can be constitutively active, inducible or repressible in the transformed microorganism. Further, the backbone of the expression vector may comprise multiple cloning sites and/or an origin of replication. Preferably, the expression vector is a plasmid.

Transformation of the microorganism with the expression vector can be effected by any acceptable method known in the art, including chemical, physical and/or enzymatic methods, such as calcium phosphate precipitation and electroporation.

Any microorganism can be used in the methods of the present invention, whether for determining antimicrobial resistance markers or as a source of donor nucleic acids, e.g., an expression library. The microorganism can be a bacterium, a fungus, e.g., a filamentous fungus, or a parasite. Exemplary bacteria include, but are not limited to, Neisseria meningitis Streptococcus pneumoniae, Streptococcus pyogenes, Moraxella catarrhalis, Bordetella pertussis, Staphylococcus aureus, Clostridium tetani, Corynebacterium diphtheria, Haemophilus influenza, Pseudomonas aeruginosa, Streptococcus agalactiae, Chlamydia trachomatis, Chlamydia pneumoniae, Helicobacter pylori, Escherichia coli, Bacillus anthracis, Yersinia pestis, Staphylococcus epidermis, Clostridium perfringens, Clostridium botulinum, Legionella pneumophila, Coxiella burnetii, Brucella spp. such as B. abortus, B. canis, B. melitensis, B. neotomae, B. ovis, B. suis, B. pinnipediae, Francisella spp. such as F. novicida, F. philomiragia, F. tularensis, Neisseria gonorrhoeae, Treponema pallidum, Haemophilus ducreyi, Enterococcus faecalis, Enterococcus faecium, Staphylococcus saprophyticus, Yersinia enterocolitica, Mycobacterium tuberculosis, Rickettsia spp., Listeria monocytogenes, Vibrio cholera, Salmonella typhi, Borrelia burgdorferi, Porphyromonas gingivalis, Klebsiella spp., Klebsiella pneumoniae.

Exemplary fungi include, but are not limited to, Dermatophytres, including Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gvpseum, Trichophyton naegnini, Trichophyton mentagrophytes, Trichophyton quinckeanum, Trichophyton rubrum, Trichophyton schoenleini, Trichophyton tonsurans, Trichophyton verrucosum, T. verrucosumvar. album, var. discoides, var. ochraceum, Trichophyton violaceum, Trichophyton faviforme; Aspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Microsporidia spp., Encephalitozoon spp., Septata intestinalis, Enterocytozoon bieneusi; Brachiola spp., Microsporidium spp., Nosema spp., Pleistophora spp., Trachipleistophora spp., Vittaforma spp., Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp., Mucor spp., Absidia spp., Mortierella spp., Cunninghamella spp., Saksenaea spp., Alternaria spp., Curvularia spp., Helminthosporium spp., Fusarium spp., Aspergillus spp., Penicillium spp., Monolinia spp., Rhizoctonia spp., Paecilomyces spp., Pithomyces spp., and Cladosporium spp.

Exemplary parasites include, but are not limited to, Plasmodium spp., such as P. falciparum, P. vivax, P. malariae and P. ovale, as well as those parasites from the Caligidae family, particularly those from the Lepeophtheirus and Caligusgenera, e.g., sea lice such as Lepeophtheirus salmonis and Caligus rogercresseyi.

Any method known in the art that is suitable for culturing the particular microorganism according to the methods of the invention can be used. For example, culturing can take place on a solid surface or in liquid suspension culture. Exemplary appropriate methods for culturing a particular microorganism are usually supplied with the microorganism ordered commercially or from one of the depository bodies, such as the American Type Culture Collection and the Deutsche Sammlung von Mikroorganismen and Zellkulturen, see also, Uruburu, 2003, Int Microbiol 6:101.

Similarly, any method known in the art that allows for the determination of microorganism growth can be used in the methods of the present invention. Preferably, the method to detellnine microorganism growth in the presence of the compound should be the same as that used to determine microorganism growth in the absence of the compound. Exemplary methods include determining cell population doubling time, measuring colony size on a plate or by absorbance in liquid cultures, or by measuring the amount of DNA production, e.g., by ³H incorporation, over time.

Any compound to which a microorganism can develop resistance can be used in the methods of the invention. Exemplary compounds include known antibiotics, antifungals and antiparasitics. For example, the following types of antibiotics are useful: aminoglycosides, ansamycins, carbacephems, carbapanems, cephalosporins, glycopeptides, lincosamides, lipopeptides, macrolides, monobactams, nitrofurans, oxazolidinones, penicillins, polypeptides, quinolones/fluoroquinolones, sulfonamides, and tetracyclines. Exemplary antibiotics include Ceftobiprole, Ceftaroline, Clindamycin, Dalbavancin, Daptomycin, Fusidic acid, Linezolid, Mupirocin (topical), Oritavancin, Tedizolid, Telavancin, Tigecycline, Carbapenems, Ceftazidime, Cefepime, Ceftobiprole, Fluoroquinolones, Piperacillin/tazobactam, Ticarcillin/clavulanic acid, Streptogramin, Tigecycline, Daptomycin, Amikacin, Gentamicin, Kanamycin, Neomycin, Netilmicin, Tobramycin, Paromomycin, Streptomycin, Spectinomycin (Bs), Geldanamycin, Herbimycin, Rifaximin, Ertapenem, Doripenem, Imipenem/Cilastatin, Primaxin, Meropenem, Dicloxacillin, Dynapen, Flucloxacillin, Mezlocillin, Methicillin, Staphcillin, Nafcillin, Oxacillin, Prostaphlin, Penicillin G, Penicillin V, Piperacillin, Penicillin G, Temocillin, Ticarcillin, Amoxicillin, Ampicillin, Azlocillin, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, Temafloxacin, Clofazimine, Dapsone, Capreomycin, Cycloserine, Ethionamide, Isoniazid, Pyrazinamide, Rifampicin (Rifampin in US), Rifabutin, Rifapentine, Streptomycin, Teixobactin;

antifungals, such as Amphotericin B, Candicidin, Filipin, Hamycin, Natamycin, Nystatin, Rimocidin, Imidazole, Triazole, Thiazole, Abafungin, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Triazole, Albaconazole, Efinaconazole, Epoxiconazole, Fluconazole, Isavuconazole, Itraconazole, Posaconazole, Propiconazole, Ravuconazole, Terconazole, Voriconazole, Thiazole, Abafungin, Allylamine, Amorolfin, Butenafine, Naftifine, Terbinafine, Echinocandin, Anidulafungin, Caspofungin, Micafungin, Echinocandin, Aurones, Benzoic acid, Ciclopirox, Flucytosine or 5-fluorocytosine, Griseofulvin, Haloprogin, Tolnaftate, Undecylenic acid, Orotomide; and antiprotozoals such as Eflornithine, Furazolidone, Melarsoprol, Metronidazole, Nifursemizone, Nitazoxanide, Ornidazole, Paromomycin sulfate, Pentamidine, Pyrimethamine, Tinidazole; antimalarials such as Quinine, Chloroquine, Amodiaquine, Pyrimethamine, Proguanil, Sulfonamides, Mefloquine, Atovaquone, Primaquine, Artemisinin and derivatives, Halofantrine, Doxycycline, and Clindamycin.

The “selected” microorganism, after at least culturing in the presence of the compound at a concentration that does not substantially inhibit wild type growth in subpart I, the survivor microorganism of subpart II and the growing (survivor) microorganism of subpart III can be collected and processed and the nucleic acids of the microorganism can be obtained and prepared for sequencing. The nucleic acids can include genomic DNA and/or extra-genomic DNA, e.g., the transformed plasmid, and/or RNA, as well as sequences corresponding to exomes, if present in the microorganism, and/or the transcriptome.

In context of the present invention, the term “sequencing” means to determine the sequence of at least one nucleic acid, and it includes any method that is used to determine the order of the bases in a strand of at least one nucleic acid. A preferred method of sequencing is high-throughput sequencing, such as next-generation sequencing or third generation sequencing.

For clarification purposes: the terms “Next Generation Sequencing” or “NGS” in the context of the present invention mean all high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods, e.g., within 1-2 weeks, preferably within 1-7 days or most preferably within less than 24 hours and allow, in principle, single cell sequencing approaches. Multiple NGS platforms which are commercially available or which are mentioned in the literature can be used in the context of the present invention, e.g., those described in detail in Zhang et al., 2011, The impact of next-generation sequencing on genomics. J. Genet Genomics 38:95-109; or in Voelkerding et al., 2009, Next generation sequencing: From basic research to diagnostics, Clinical chemistry 55:641-658. Non-limiting examples of such NGS technologies/platforms are
1) The sequencing-by-synthesis technology known as pyrosequencing implemented, e.g., in the GS-FLX 454 Genome Sequencer™ of Roche-associated company 454 Life Sciences (Branford, Conn.), first described in Ronaghi et al., 1998, A sequencing method based on real-time pyrophosphate, Science 281:363-365. This technology uses an emulsion PCR in which single-stranded DNA binding beads are encapsulated by vigorous vortexing into aqueous micelles containing PCR reactants surrounded by oil for emulsion PCR amplification. During the pyrosequencing process, light emitted from phosphate molecules during nucleotide incorporation is recorded as the polymerase synthesizes the DNA strand.
2) The sequencing-by-synthesis approaches developed by Solexa (now part of Illumina Inc., San Diego, Calif.) which is based on reversible dye-terminators and implemented, e.g., in the Illumina/Solexa Genome Analyzer™ and in the Illumina HiSeq 2000 Genome Analyzer™. In this technology, all four nucleotides are added simultaneously into oligo-primed cluster fragments in flow-cell channels along with DNA polymerase. Bridge amplification extends cluster strands with all four fluorescently labeled nucleotides for sequencing.
3) Sequencing-by-ligation approaches, e.g., implemented in the SOLid™ platform of Applied Biosystems (now Life Technologies Corporation, Carlsbad, Calif.). In this technology, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. As a second example, the Polonator™ G.007 platform of Dover Systems (Salem, N.H.) also employs a sequencing-by-ligation approach by using a randomly arrayed, bead-based, emulsion PCR to amplify DNA fragments for parallel sequencing.
4) Single-molecule sequencing technologies such as, e.g., implemented in the PacBio RS system of Pacific Biosciences (Menlo Park, Calif.) or in the HeliScope™ platform of Helicos Biosciences (Cambridge, Mass.). The distinct characteristic of this technology is its ability to sequence single DNA or RNA molecules without amplification, defined as Single-Molecule Real Time (SMRT) DNA sequencing. For example, HeliScope uses a highly sensitive fluorescence detection system to directly detect each nucleotide as it is synthesized. A similar approach based on fluorescence resonance energy transfer (FRET) has been developed from Visigen Biotechnology (Houston, Tex.). Other fluorescence-based single-molecule techniques are from U.S. Genomics (GeneEngine™) and Genovoxx (AnyGene™).
5) Nano-technologies for single-molecule sequencing in which various nanostructures are used which are, e.g., arranged on a chip to monitor the movement of a polymerase molecule on a single strand during replication. Non-limiting examples for approaches based on nano-technologies are the GridON™ platform of Oxford Nanopore Technologies (Oxford, UK), the hybridization-assisted nano-pore sequencing (HANS™) platforms developed by Nabsys (Providence, R.I.), and the proprietary ligase-based DNA sequencing platform with DNA nanoball (DNB) technology called combinatorial probe-anchor ligation (cPAL™).
6) Electron microscopy based technologies for single-molecule sequencing, e.g., those developed by LightSpeed Genomics (Sunnyvale, Calif.) and Halcyon Molecular (Redwood City, Calif.)
7) Ion semiconductor sequencing which is based on the detection of hydrogen ions that are released during the polymerization of DNA. For example, Ion Torrent Systems (San Francisco, Calif.) uses a high-density array of micro-machined wells to perform this biochemical process in a massively parallel way. Each well holds a different DNA template. Beneath the wells is an ion-sensitive layer and beneath that a proprietary Ion sensor.

Other sequencing methods useful in the context of the invention include tunneling currents sequencing (Xu et al., 2007, The electronic properties of DNA bases, Small 3:1539-1543, Di Ventra, 2013, Fast DNA sequencing by electrical means inches closer, Nanotechnology 24:342501). Particularly preferable next-generation sequencing (NGS) methodologies include Illumina, IONTorrent and NanoPore sequencing.

Preferably, DNA and RNA preparations serve as starting material for NGS. Such nucleic acids can be easily obtained from the microorganisms. Although nucleic acids extracted can be highly fragmented, they are nonetheless suitable for NGS applications.

In embodiments of the present invention where the genomic nucleic acids of the microorganism contain introns, targeted NGS methods for exome sequencing can be used, for review see, e.g., Teer and Mullikin, 2010, Human Mol Genet 19:R145-51. Many of these methods (described, e.g., as genome capture, genome partitioning, genome enrichment, etc.) use hybridization techniques and include array-based (e.g., Hodges et al., 2007, Nat Genet 39:1522-1527) and liquid-based (e.g., Choi et al., 2009, Proc Natl Acad Sci USA 106:19096-19101) hybridization approaches. Commercial kits for DNA sample preparation and subsequent exome capture are also available: for example, Illumina Inc. (San Diego, Calif.) offers the TruSee™ DNA Sample Preparation Kit and the Exome Enrichment Kit TruSee™ Exome Enrichment Kit.

Once the nucleic acids have been sequenced, the resulting sequences can be compared to a reference sequence, e.g., the sequence of the same microorganism prior to any exposure to the compound to determine differences between the sequenced nucleic acids and the reference, and thus identify the nucleic acid-encoded biomarker(s) providing for the ability of the microorganism to grow in the presence of the compound at a concentration that prevents the wild-type microorganism from growing. In an embodiment, the reference sequence can be the sequence of the corresponding genomic (DNA) nucleic acids from the wild-type microorganism or can be a consensus sequence of several strains the microorganism that are not resistant to the compound. The reference sequence can be contained in one or more databases comprising the genetic information preferably from multiple species.

In certain embodiments of the invention, in order to reduce the number of false positive findings in detecting and comparing sequences, it is preferred to determine/compare the sequences in replicates. Thus, it is preferred that nucleic acid sequences of the cultured/selected microorganisms be determined twice, three times or more. For example, by determining the variations between replicates of a sample, the expected rate of false positive (FDR) mutations as a statistical quantity can be estimated. Technical repeats of a sample should generate identical results and any detected mutation in this “same vs. same comparison” is a false positive. Furthermore, various quality related metrics (e.g., coverage or SNP quality) may be combined into a single quality score using a machine learning approach.

In context of the present invention, the term “database” relates to an organized collection of data, preferably as an electronic filing system. In an embodiment, a sequence database is a type of database that is composed of a collection of computerized (“digital”) nucleic acid sequences, protein sequences, or other polymer sequences stored on a computer. Preferably, the database is a collection of nucleic acid sequences, i.e., the genetic information from a number of species/strains. The genetic information can be derived from the genome and/or the exome and/or the transcriptome of a species. Exemplary nucleic acid databases useful in the present invention include, but are not limited to, International Nucleotide Sequence Database (IVSD), DNA Data Bank of Japan (National Institute of Genetics), EMBL (European Bioinformatics Institute), GenBank (National Center for Biotechnology Information), Bioinformatic Harvester, Gene Disease Database, SNPedia, CAMERA Resource for microbial genomics and metagenomics, EcoCyc (a database that describes the genome and the biochemical machinery of the model organism E. coli K-12), Ensembl (provides automatic annotation databases for human, mouse, other vertebrate and eukaryote genomes) Ensembl Genomes (provides genome-scale data for bacteria, protists, fungi, plants and invertebrate metazoa, through a unified set of interactive and programmatic interfaces (using the Ensembl software platform)), Exome Aggregation Consortium (ExAC) (exome sequencing data from a wide variety of large-scale sequencing projects (Broad Institute)), PATRIC (PathoSystems Resource Integration Center), MGI Mouse Genome (Jackson Laboratory), JGI Genomes of the DOE-Joint Genome Institute (provides databases of many eukaryote and microbial genomes), National Microbial Pathogen Data Resource (a manually curated database of annotated genome data for the pathogens Campylobacter, Chlamydia, Chlamydophila, Haemophilus, Listeria, Mycoplasma, Neisseria, Staphylococcus, Streptococcus, Treponema, Ureaplasma and Vibrio), RegulonDB (a model of the complex regulation of transcription initiation or regulatory network of the cell E. coli K-12), Saccharomyces Genome Database (genome of the yeast model organism), The SEED platform (includes all complete microbial genomes, and most partial genomes, the platform is used to annotate microbial genomes using subsystems), WormBase ParaSite (parasitic species), UCSC Malaria Genome Browser (genome of malaria causing species (Plasmodium falciparum and others)), INTEGRALL (database dedicated to integrons, bacterial genetic elements involved in the antibiotic resistance), VectorBase (NIAID Bioinfonnatics Resource Center for Invertebrate Vectors of Human Pathogens), EzGenome, comprehensive information about manually curated genome projects of prokaryotes (archaea and bacteria), GeneDB (Apicomplexan Protozoa, Kinetoplastid Protozoa, Parasitic Helminths, Parasite Vectors as well as several bacteria and viruses), GEAR DB (GEnetic Antibiotic Resistance and Susceptibility Database), EuPathDB (eukaryotic pathogen database resources includes amoeba, fungi, plasmodium, trypanosomatids etc.); The 1000 Genomes Project (providing the genomes of more than a thousand anonymous participants from a number of different ethnic groups), Personal Genome Project (providing human genomes).

In embodiments where the expression vector, when transformed into a microorganism gives that microorganism resistance to a compound, expresses more than one nucleic acid-encoded molecule, the nucleic acid inserted into the expression vector can be fragmented and used as starting material to make a (sub)-library which is then used in subpart III of the method.

The term “genome” relates to the total amount of genetic information in the chromosomes of an organism or a cell. For organisms that do not have chromosomes, “genome” relates to the total amount of DNA-based or RNA-based genetic information. The term “exome” refers to part of the genome of an organism formed by exons, which are coding portions of expressed genes. Where an organism does not have exons/introns, “exome” relates to the nucleic acids that encode molecules, such as proteins and other nucleic acids, e.g., RNA. The exome provides the genetic blueprint used in the synthesis of proteins and other functional gene products. It is the most functionally relevant part of the genome and, therefore, it is most likely to contribute to the phenotype of an organism. The term “transcriptome” relates to the set of all RNA molecules, including mRNA, rRNA, tRNA, and other non-coding RNA produced in one cell or a population of cells. In context of the present invention the transcriptome means the set of all RNA molecules produced in one cell, a population of cells, or all cells of a given organism at a certain time point.

The term “genetic material” includes isolated nucleic acid, either DNA or RNA, a section of a double helix, a section of a chromosome, or an organism's or cell's entire genome, in particular its exome or transcriptome.

According to the invention, “nucleic acid” is preferably deoxyribonucleic acid (DNA) or ribonucleic acid (RNA). Nucleic acids include genomic DNA, cDNA, mRNA, recombinantly produced and chemically synthesized molecules. A nucleic acid may be present as a single-stranded or double-stranded and linear or covalently circularly closed molecule, as well as mixtures thereof. A nucleic acid can be isolated. Preferably, the nucleic acid is a free circulating DNA and/or RNA molecule. In one embodiment, the term “nucleic acid” is also understood to mean “nucleic acid sequence”. Further, prior to sequencing, the nucleic acids can be processed, for example, enriched or amplified. In cases where the nucleic acid obtained from the sample is RNA, the RNA can be reverse transcribed into DNA for sequencing or the RNA itself can be sequenced.

The term “mutation” refers to a change of or difference in the nucleic acid sequence (nucleotide substitution, addition or deletion) compared to a reference.

In an embodiment, the method can further comprise testing at least one identified nucleic acid-encoded antimicrobial resistance biomarker of the panel of biomarkers identified in a microorganism for a particular compound for the ability to confer antimicrobial resistance to the compound in a microorganism that does not have the biomarker. Preferably, the testing comprises culturing a microorganism recombinantly expressing at least one identified nucleic acid-encoded antimicrobial resistance biomarker in the presence of the compound in an amount/concentration that substantially inhibits wild-type growth of the microorganism not having the biomarker. Where the recombinant microorganism grows in the presence of the compound at the concentration known to be inhibitory, the encoded product of the biomarker confers resistance to the compound.

The present invention is directed to a plurality of nucleic acid-encoded antimicrobial resistance biomarkers of a microorganism for a particular compound, which biomarkers preferably have been identified by the method described herein, and which indicate that the microorganism is resistant to the compound, i.e., can grow substantially as well in the presence of the compound as in the absence, at concentrations what would substantially inhibit the growth of a corresponding microorganism not having the biomarker.

The present invention is also directed to a method for determining the presence of a microorganism resistant to a particular compound in a host organism, comprising detecting at least one nucleic acid-encoded antimicrobial resistance biomarker, preferably identified by the method disclosed herein in a microorganism obtained from a biological sample of the host organism. The method can comprise isolating a microorganism from a biological sample obtained from a host organism and determining, e.g., by sequencing, whether the isolated microorganism comprises a nucleic acid-encoded antimicrobial resistance biomarker previously identified to provide resistance to the particular compound. Where the biomarker is present in the microorganism, the microorganism is resistant to the particular compound.

The terms “host organism”, “subject”, “individual”, “organism” or “patient” are used interchangeably and preferably relate to vertebrates, preferably mammals. For example, mammals in the context of the present invention are humans, non-human primates, domesticated animals such as dogs, cats, sheep, cattle, goats, pigs, horses etc., laboratory animals such as mice, rats, rabbits, guinea pigs, etc. as well as animals in captivity such as animals of zoos. The term “animal” also includes humans. Preferably, the terms “subject”, “individual”, “organism” or “patient” refer to male and female mammals, in particular male and female humans.

The term “in vivo” relates to the situation in a subject.

As used herein, “biological sample” includes any biological sample obtained from a host organism. Examples of such biological samples include blood, smears of cells, sputum, bronchial aspirate, urine, stool, bile, gastrointestinal secretions, lymph fluid, bone marrow, organ aspirates and tissue biopsies, including punch biopsies. Optionally, the biological sample can be obtained from a mucous membrane of the patient.

The present invention is also directed to a method for screening/testing microorganisms for resistance to a particular compound. In an embodiment, the method comprises screening a microorganism for the presence of at least one nucleic acid-encoded antimicrobial resistance biomarker of the panel of biomarkers identified in the same type or similar microorganism for a particular compound. Preferably, screening comprises sequencing the nucleic acids of the microorganism to determine the presence of the at least one nucleic acid-encoded antimicrobial resistance biomarker in the microorganism. Where the biomarker is present in the nucleic acids of the microorganism, the microorganism is resistant to the compound.

The present invention is described in detail by the FIGURE and example below, which are used only for illustration purposes and are not meant to be limiting. Owing to the description and the examples, further embodiments which are likewise included in the invention are accessible to the skilled worker.

### 5. BRIEF DESCRIPTION OF THE FIGURES

FIG. 1 is a schematic exemplary representation of the method for providing a panel of nucleic acid-encoded antimicrobial resistance biomarkers, including optional steps, in order to identify the panel of antimicrobial resistance biomarkers in accordance with the present invention.

### 6. EXAMPLE

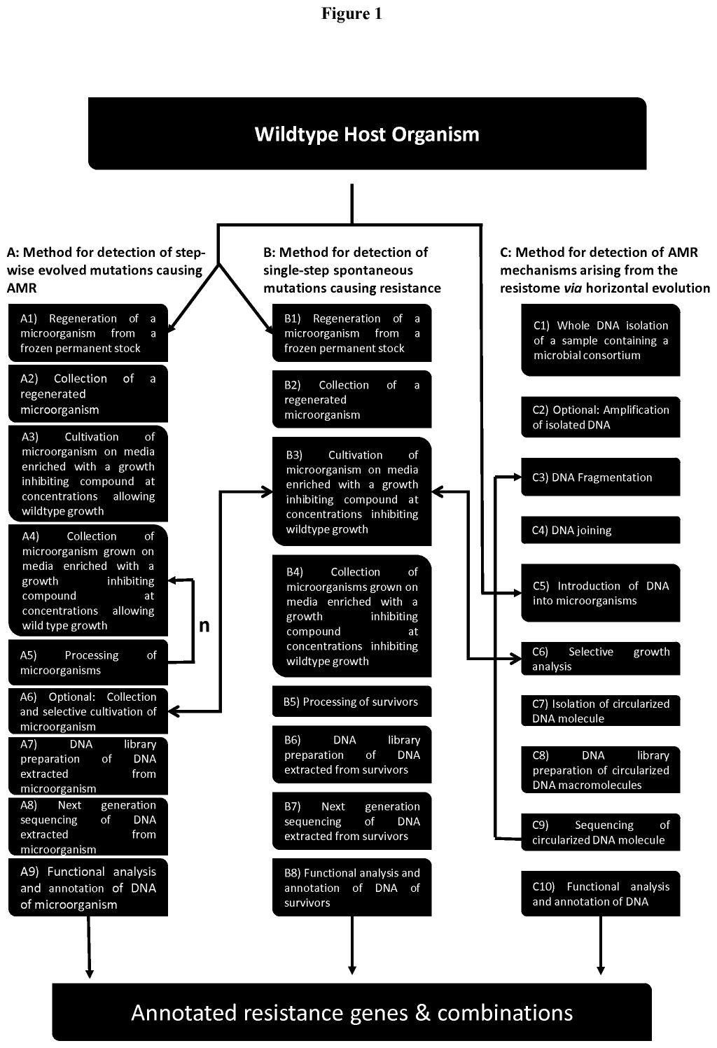

A summary of the elements—and their interconnections—of the method for providing a panel of nucleic acid-encoded antimicrobial resistance (AMR) biomarkers in accordance with the present invention is depicted in FIG. 1. In all cases, a suitable wild-type host microorganism is used; however, non-standard phenotypes can also be used.

Method A depicts detecting nucleic acid-encoded biomarkers caused by a stepwise evolved resistance mechanism resulting from serial passaging (see loop from A5 to A4) of the microorganism at concentrations of a compound below the minimum inhibitory concentration (MIC) of the compound, i.e., at concentrations that do not substantially inhibit wild-type growth of the microorganism. Optional parallel plating at higher concentrations (equal to or greater than MIC, i.e., at concentrations that substantially inhibit wild-type growth of the microorganism) can be used to monitor for resistance to the compound (indicated by connection line from A6 to B3).

Method B depicts detecting nucleic acid-encoded biomarkers caused by a single-step spontaneously evolved resistance mechanism, resulting from culturing the microorganism with the compound at concentrations above the minimum inhibitory concentration (MIC), i.e., at concentrations that substantially inhibit wild-type growth of the microorganism. Only mutants acquiring spontaneous “survivor-mutations”, which result in a large shift in MIC, will be resistant against higher concentrations of the compound and will be capable of growth. These mutations can be different to those which are detected by Method A because of the difference in selection pressures and fitness (Westhoff et al., 2017, ISME J. 11:1168-1178).

Method C depicts detecting nucleic acid-encoded biomarkers caused by horizontal transfer. As depicted, metagenomic DNA, optionally obtained from a pooled resistome of varying density and genetic complexity serves as template for the generation of a plasmid library, which library is transformed into the microorganism with subsequent monitoring of transformed microbial growth of the mutants in the presence of the compound at concentrations above the minimum inhibitory concentration, i.e., at concentrations that substantially inhibit wild-type growth of the microorganism. Microbial growth may indicate a potentially unknown mechanism due to the transformed metagenomic DNA provided to the microorganism allowing for resistance against the compound.

After the cultivation of the (now mutant/transformed) microorganism in the presence of the compound, the nucleic acids of the microorganism are sequenced and differences between the sequence and a reference sequence are determined, which differences, e.g., in encoded proteins or other nucleic acids including mRNA and ribosome sequences, are biomarkers for resistance to the compound in that microorganism. Specific to method C, after cultivation, subsequent plasmid sequencing thereby identifies the nucleic acid-encoded biomarker(s) causing the resistance. In case that more than biomarker is encoded by the plasmid, the plasmid can be fragmented and steps C3 to C9 are repeated (see loop from C9 to C3).

Carrying out the three methods (A-C) allows for the identification of a (comprehensive) plurality (panel) of nucleic acid-encoded antimicrobial resistance biomarkers for a particular compound in a microorganism, which biomarkers are caused not only by de novo mechanisms but also by those nucleic acids acquired by horizontal gene transfer from related and/or unrelated donor microorganisms. These identified biomarkers can then be annotated to a particular gene and functionally analyzed, e.g., confirming their identity as a resistance biomarker for a particular compound by expressing such biomarkers in the wild-type microorganism and testing for growth ability in the presence of the compound.

What is claimed:

1. A method for providing a panel of nucleic acid-encoded antimicrobial resistance (AMR) biomarkers of a microorganism, wherein the panel of AMR biomarkers relates to a compound having antimicrobial activity, the method comprising:
I (a) culturing the microorganism in the presence of the compound, wherein the compound is present in an amount that does not substantially inhibit wild type growth of the microorganism under the same culture conditions absent the compound, and
(b) determining the genetic profile of the cultured microorganism obtained in step (a);
II (c)(i) culturing the microorganism in the presence of the compound, wherein the compound is present in an amount that substantially inhibits wild type growth of the microorganism under the same culture conditions absent the compound, and (ii) selecting a cultured microorganism whose growth in the presence of the compound is substantially the same as in the absence of the compound, and
(d) determining the genetic profile of the selected microorganism obtained in step (c); and
III (e)(i) culturing the microorganism in the presence of the compound, wherein the compound is present in an amount that substantially inhibits wild type growth of the microorganism under the same culture conditions absent the compound, and (ii) selecting a cultured microorganism whose growth in the presence of the compound is substantially the same as in the absence of the compound, wherein the microorganism is a recombinant microorganism, and
(f) determining the genetic profile of the selected microorganism obtained in step (e);
wherein the genetic profiles determined in steps (b), (d), and (f) are each compared to a reference genetic profile of the microorganism to identify a panel of nucleic acid-encoded antimicrobial resistance (AMR) biomarkers relating to the compound.

2. The method according to claim 1, wherein the genetic profile is determined by sequencing the genomic and optionally the extra-genomic nucleic acids of the microorganisms obtained in steps (a), (c), and (e), optionally by next-generation sequencing.

3. The method according to claim 2, wherein the extra-genomic nucleic acids are naturally occurring in the microorganism or are not naturally occurring, for example, an artificial plasmid transformed into the microorganism.

4. The method according to any one of claims 1 to 3, wherein after step (a) and prior to step (b), the method further comprises (i) culturing the microorganism in the presence of the compound in an amount that substantially inhibits wild type growth of the microorganism under the same culture conditions absent the compound, and (ii) selecting a cultured microorganism whose growth in the presence of the compound is substantially the same as in the absence of the compound.

5. The method according to any one of claims 1 to 4, wherein the steps (i) and (ii) are repeated at least once.

6. The method according to any one of claims 1 to 5, wherein the recombinant microorganism is genetically modified.

7. The method according to claim 6, wherein the genetically modified recombinant microorganism comprises an expression library.

8. The method according to claim 7, wherein the expression library is derived from a different strain of the same microorganism or is derived from a different microorganism or is derived from a consortium of microorganisms.

9. The method according to any one of claims 1 to 8, wherein the microorganism is a bacterium, a fungus, or a parasite, preferably a bacterium.

10. The method according to any one of claims 1 to 9, wherein the growth of the microorganism is measured by cell doubling time.

11. The method according to any one of claims 1 to 10, wherein culturing takes place on a solid surface.

12. The method according to any one of claims 1 to 11, wherein the compound is an antibiotic or antifungal compound.

13. The method according to any one of claims 1 to 12, wherein the method further comprises testing at least one biomarker of the identified panel of biomarkers for the ability to confer antimicrobial resistance to the compound.

14. The method according to claim 13, wherein the testing comprises culturing a microorganism recombinantly expressing the at least one biomarker in the presence of the compound in an amount that substantially inhibits wild-type growth of the microorganism not having the biomarker.

15. A panel of nucleic acid-encoded antimicrobial resistance biomarkers identified by the method according to any one of claims 1 to 14.

16. A method for determining the presence of a microorganism resistant to the compound in a host organism, comprising detecting at least one nucleic acid-encoded antimicrobial resistance biomarker identified by the method according to any one of claims 1 to 14 in a microorganism obtained from the host organism.

17. The method according to claim 16, wherein the host organism is a mammal, preferably a human.
